(19) United States
(12) Patent Application Publication
(10) Pub. No.: US 2024/0058479 A1
(43) Pub. Date: Feb. 22, 2024

(54) BIODEGRADABLE RADIOPAQUE MICROSPHERE

(71) Applicant: Varian Medical Systems, Inc., Palo Alto, CA (US)

(72) Inventors: Donald Bejleri, Round Rock, TX (US); Theodore Ronald Jackson, JR., Ellicott City, MD (US)

(21) Appl. No.: 18/451,174

(22) Filed: Aug. 17, 2023

Related U.S. Application Data
(60) Provisional application No. 63/371,963, filed on Aug. 19, 2022.

(51) Int. Cl. A61K 49/04

(52) U.S. Cl. CPC A61K 49/04; A61B 2090/3966

(57) ABSTRACT

A microsphere includes a radiopaque material, and a biodegradable material such that the microsphere can be detected using medical imaging and biodegrade after being administered to a patient.

### CROSS REFERENCE TO RELATED APPLICATIONS

This application claims the benefit of U.S. Provisional Patent Application No. 63/371,963, filed Aug. 19, 2022, which is hereby incorporated by reference for all purposes as if fully set forth herein.

### TECHNICAL FIELD

The present disclosure relates to microspheres for imaging used during radioembolization treatments. More specifically, the present disclosure relates to biodegradable radiopaque microspheres used to monitor radioembolization implants and dosimetry during treatment and methods of making the biodegradable radiopaque microspheres.

### BACKGROUND

Radioembolization is a type of internal radiation therapy that delivers targeted radiation doses to treat various cancers, including unresectable hepatic malignancies from, for example, primary colorectal cancer, metastatic colorectal carcinoma, and hepatic metastases from other primary tumor types. Radioembolization involves administering patients with micron-sized glass or polymer microspheres loaded with a radioisotope (most commonly yttrium-90 (i.e., Y-90)) through minimally invasive injection. This treatment provides a short-range and strong beta (β) emission to tissue to destroy or kill the cancer cells. Radioembolization therapy has been successful in treating cancers especially in cases where surgical resection or organ transplantation is not possible or more invasive.

However, microspheres used in conventional radioembolization are limited by poor evaluation of dose (dosimetry) during treatment and cannot be visualized using fluoroscopy, computed tomography (CT), or cone-beam computed tomography (CBCT) imaging. Medical professionals rely on post-operative evaluation of dose via imaging using single-photon emission computerized tomography (SPECT), positron emission tomography (PET), or other methods with the consequence that dosimetry is expensive or inaccessible and is difficult to quantify in clinically meaningful dose-to-target terms. If the treatment dose was determined to be too low after treatment, patients need to wait for future treatments to receive additional radiation doses to adjust the total dosage. Therefore, in-room evaluation of radioembolization dose at the time of the procedure is desirable to improve dosimetry, radioisotope delivery, and treatment outcomes.

Additionally, incorporating conventional (i.e., non-biodegradable) radiopaque elements result in non-degradable microspheres which are retained in the patient's body during and beyond treatment. Because patients commonly need multiple radioembolization treatments to achieve cancer remission, conventional radiopaque microspheres will build up in the implanted region of the patient from repeated treatments and make it increasingly difficult to effectively evaluate any additional treatment dose by obscuring surrounding anatomy during follow-up imaging.

### SUMMARY OF THE DISCLOSURE

In various embodiments of the present disclosure, microspheres and methods of making the microspheres are provided so that radiopaque microspheres can be used with medical imaging to monitor progress of a radioembolization treatment during the procedure. Being able to provide this information is an improvement over existing and traditional methods of treatment by providing real-time feedback regarding the dose and delivery of radioactive microspheres during treatment without causing patient retention of microspheres that are used only for imaging. This real-time information can improve the effectiveness of the radioembolization treatment and can reduce the likelihood that subsequent treatments are performed.

According to an embodiment, a microsphere includes a radiopaque material and a biodegradable material.

In one aspect, the radiopaque material is a nanoparticle.

In another aspect, the biodegradable material is chemically bound to the radiopaque material.

In another aspect, the radiopaque material is encapsulated by the biodegradable material.

In another aspect, the biodegradable material is coated by the radiopaque material.

In another aspect, the biodegradable material includes at least one of gelatin, alginate, chitosan, hyaluronic acid, collagen, cellulose, fibrinogen, fibrin, silk, elastin, hydroxyapatite, decellularized matrix, heparin, agarose, natural polysaccharides, albumin, gelatin methacrylate, triacyl gelatin, and hyaluronic acid methacrylate.

In another aspect, the biodegradable material includes at least one of poly(L-lactide) (PLLA), poly(lactic-co-glycolic acid) (PLGA), polyethylene glycol (PEG), polyurethane (PU), and polyvinyl alcohol (PVA).

In another aspect, the radiopaque material includes at least one of tantalum, gold, barium, strontium, gallium or iodine which may be in the form of pure metals, metal oxides, alloys, compounds, or any other suitable chemical formulation.

According to an embodiment, a method of making a microsphere comprises linking a biodegradable material with a radiopaque material.

According to an embodiment, a method of making a microsphere comprises encapsulating a radiopaque material with a biodegradable material.

According to an embodiment, a method of making a microsphere comprises coating a biodegradable material with a radiopaque material.

According to an embodiment, a method of making a microsphere includes producing radiopaque material, combining the radiopaque material with a biodegradable material, and finishing processing biodegradable radiopaque microspheres.

In an aspect, the radiopaque material is provided by synthesizing radiopaque nanoparticles through addition of a surfactant to a raw radiopaque material.

In an aspect, the radiopaque material is provided by creating a radiopaque material and a biodegradable linker complex.

In an aspect, the linker complex includes at least one of an amine, a carboxyl group, a peptide, a protein or functional polymer, and is biodegradable.

In an aspect, the radiopaque material is provided as a metal-organic complex.

In an aspect, the finish processing includes obtaining biodegradable radiopaque microspheres of uniform size distribution and target properties.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

The features and advantages of the present disclosures will be more fully disclosed in, or rendered apparent by the following detailed descriptions of example embodiments. The detailed descriptions of the example embodiments are to be considered together with the accompanying drawings wherein like numbers refer to like parts and further wherein:

FIG. 1 is a process flow diagram of a method of making a biodegradable radiopaque microsphere according to the present disclosure.

FIG. 2 is a process flow diagram of methods of making a radiopaque material according to the present disclosure.

FIG. 3 is a process flow diagram of methods of making microspheres with a radiopaque material according to the present disclosure.

FIG. 4 is a process flow diagram of methods to obtain microspheres of uniform size distribution and target properties according to the present disclosure.

FIG. 5 is a diagram of a biodegradable radiopaque microsphere according to an embodiment of the present disclosure.

FIG. 6 is a diagram of a biodegradable radiopaque microsphere according to another embodiment of the present disclosure.

FIG. 7 is a diagram of a biodegradable radiopaque microsphere according to another embodiment of the present disclosure.

### DETAILED DESCRIPTION

The description of the preferred embodiments is intended to be read in connection with the accompanying drawings, which are to be considered part of the entire written description of these disclosures. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

It should be understood, however, that the present disclosure is not intended to be limited to the particular forms disclosed. Rather, the present disclosure covers all modifications, equivalents, and alternatives that fall within the spirit and scope of these exemplary embodiments. The terms “couple,” “coupled,” “operatively coupled,” “operatively connected,” and the like should be broadly understood to refer to connecting devices or components together either mechanically, electrically, wired, wirelessly, or otherwise, such that the connection allows the pertinent devices or components to operate (e.g., communicate) with each other as intended by virtue of that relationship.

In the present disclosure the singular forms “a,” “an,” and “the” include the plural reference, and reference to a particular numerical value includes at least that particular value, unless the context clearly indicates otherwise. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.” It is intended that any component, element, attribute, or step that is positively recited herein may be explicitly excluded in the claims, whether such components, elements, attributes, or steps are listed as alternatives or whether they are recited in isolation.

The present disclosure relates to biodegradable radiopaque microspheres. Biodegradable radiopaque microspheres can be administered alone or can be mixed with radioembolization microspheres prior to administration so that the administered batch of microspheres can be visualized during treatment. Moreover, the biodegradable radiopaque microspheres will biodegrade and be resorbed by the patient's tissue over time to reduce or eliminate artifacts or interference of future imaging studies. In this way, disease treatment can be more easily monitored across multiple doses or treatments.

To provide in-room imaging during a radioembolization procedure, a component that permits imaging can be incorporated during treatment and correlated to the radioactive dose delivered via the radioembolization microspheres. Including a radiopaque element delivered during the treatment facilitates in-room imaging during treatment.

A biodegradable radiopaque microsphere would overcome the issues described above by degrading between treatments, allowing for in-room dosimetry of each radioembolization treatment while limiting imaging signal interference across treatments. The biodegradable radiopaque microsphere is meant as a complimentary particle to the radioactive treatment microsphere, where both particles have similar characteristics related to particle flow in vasculature, such as density and surface charge/binding, which provides correlation of a radiopaque signal to the dose distribution. The combination of a radioactive Y-90 treatment microsphere with a biodegradable radiopaque microsphere overcomes limitations of existing radioembolization therapies by providing in-room dose evaluation, degradable imaging interference between treatments, and matching radiotherapy dosages. In addition, biodegradable radiopaque microsphere fabrication can be a low cost and low complexity synthesis process, which can incorporate tunable properties to match desired treatment particle flow characteristics.

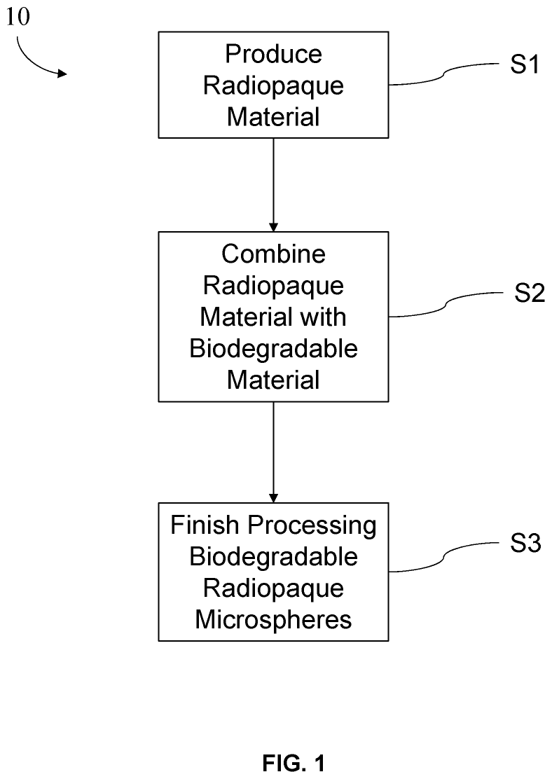

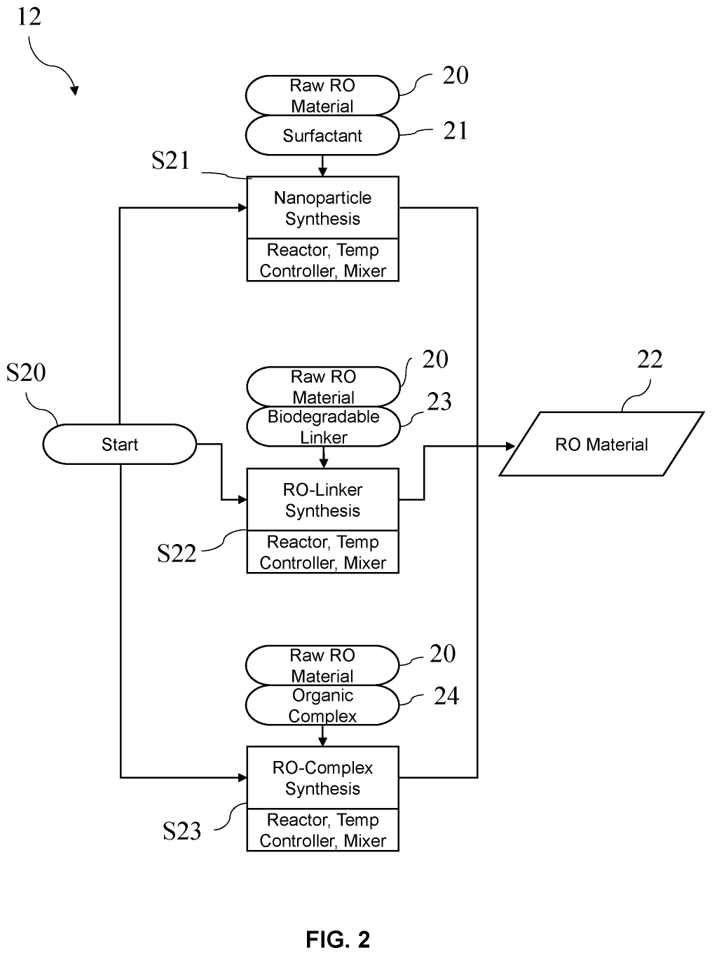

Fabrication of biodegradable radiopaque microspheres can be produced using various methods as further described below. In one example, as shown in FIG. 1, a method 10 can be used to produce biodegradable radiopaque microspheres. The method 10 can include three steps. The three steps can include, (S1) Produce Radiopaque Material; (S2) Combine the Radiopaque Material with Biodegradable Material; and (S3) Finishing Processing of the Biodegradable Radiopaque Microspheres. At step S1, radiopaque (RO) material can be produced. The RO material, when incorporated into or with a microsphere, can allow the microsphere to be visualized during treatment. Various methods of producing the RO material can be used. Further description of example methods and processes that can be used to produce the RO material at step S1 are described with reference to FIG. 2 below.

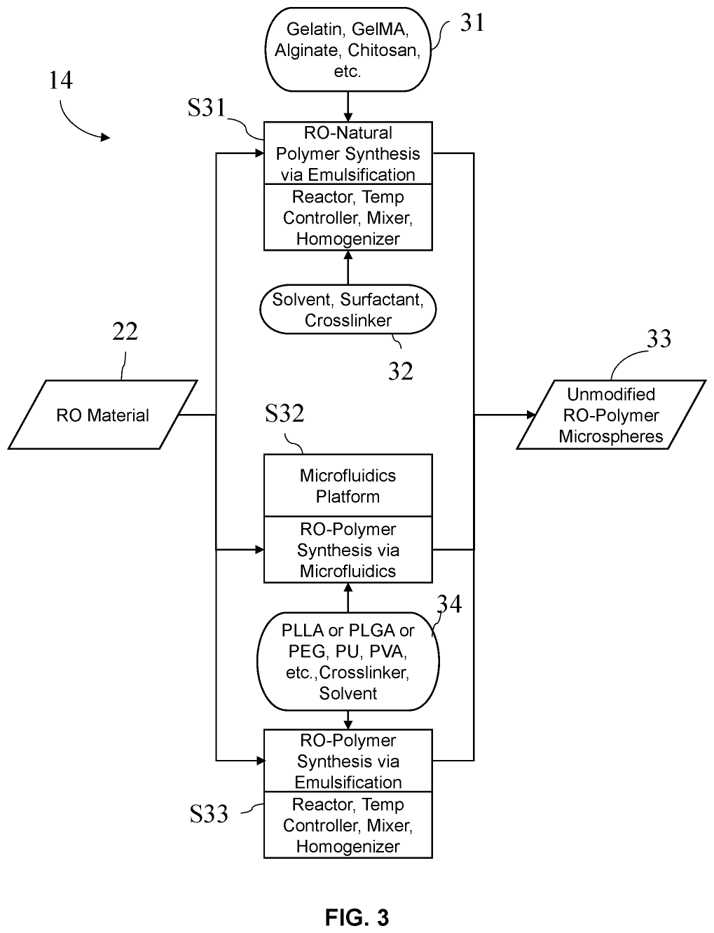

At step S2, the RO material that is produced at step S1 can be combined with biodegradable material to form a biodegradable microsphere. Various processes and methods can be used at step S2 to define a microsphere that incorporates the RO material from step S1. Further description of example processes and/or methods that can be used at step S2 are described below with reference to FIG. 3. In other examples, other processes and/or methods can also be used.

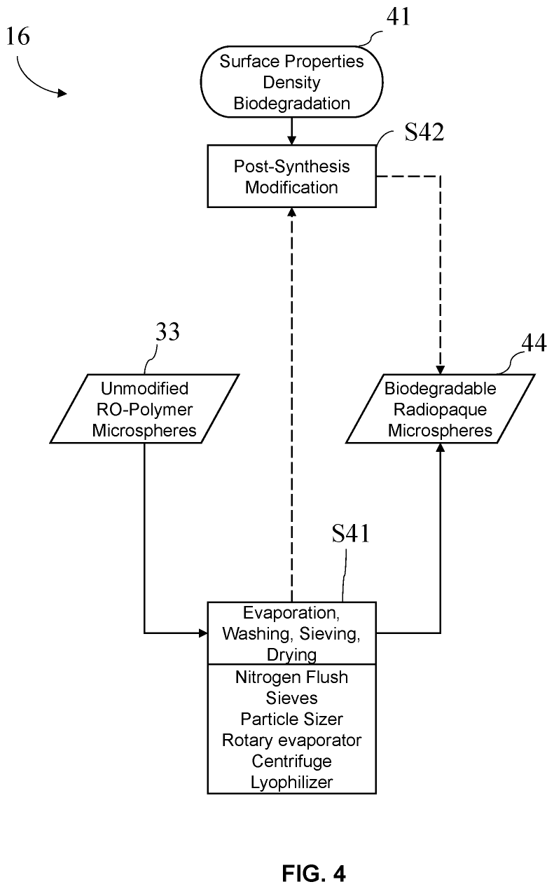

The method 10 can continue to step S3. At step S3, the finishing processes can be performed on the biodegradable radiopaque microspheres that were produced at step S2. The finishing processes of step S3 can include quality control processes, rinsing, sieving, or other processes that can provide a group, volume and/or quantity of biodegradable radiopaque microspheres that are in a state to be used during a treatment procedure. Further description of example processes and/or methods that can be performed at step S3 are described below with reference to FIG. 4. In other examples, other finishing processes and/or methods can be used.

While not shown in method 10, other steps can be performed in addition to the steps shown. For example, once biodegradable radiopaque microspheres are processed using method 10, the biodegradable radiopaque microspheres can then be mixed in desired proportions with radioactive microspheres prior to treatment and administration.

In FIG. 2, example methods of producing RO material are shown. The method 12 includes three pathways or optional sub-methods that can be used to produce a radiopaque material. Any of these three pathways or sub-methods can be used at step S1 of method 10 described above. Radiopaque material can be produced in a manner that can be bound or incorporated into a biodegradable microsphere. For example, RO materials can include at least one of tantalum, gold, barium, strontium, gallium, iodine, or any other suitable bioinert material that can be recognized in vivo imaging. These materials can be provided as a fine powder, nanoparticles, or any other suitable physical formulation. They can be pure metals, metal oxides, alloys, compounds, or any other suitable chemical formulation.

FIG. 2 shows three examples of production methods to create a RO material 22 for microsphere incorporation. One example, shown as the top path in FIG. 2, is to synthesize radiopaque material nanoparticles through addition of a surfactant or another chemical to a raw radiopaque material to form nanoparticles of the raw radiopaque material. After start at S20, raw RO material 20 can be mixed with a surfactant 21 in step S21 to generate RO material 22. A reactor with temperature and mixing control can be used in this step. As further discussed below with respect to FIG. 3, the synthesized RO material 22 as nanoparticles can then be incorporated into microspheres, through direct binding of nanoparticle surface groups to a microsphere material, coating a surface of a microsphere material, encapsulation within the microsphere during formation, or post-synthesis loading.

These formations are similar to loading drugs or other materials into microspheres, where the nanoparticles can coat the surface of the microsphere (for cases of direction conjugation) or be embedded within the microsphere matrix (for all three cases described above). For direct conjugation of the nanoparticle into or on the surface of the microsphere, conjugation chemistry between the nanoparticle functional groups and the microsphere functional groups can be used to generate covalent bonding between the microsphere and nanoparticle. These functional groups can include hydrocarbons, halogenated groups, groups containing oxygen, groups containing nitrogen, groups containing sulfur, groups containing phosphorous, groups containing metals, or groups containing any other common chemical moiety on the microspheres and/or nanoparticles. Alternately, nanoparticles can be loaded into the microparticle (either via encapsulation during microsphere formation or through post-synthesis loading) through physical interactions driven by charged group interactions, hydrophobic/hydrophilic interactions, hydrogen bonding, pi-pi stacking, or weaker forces such as Van der Waals. These interactions do not form covalent bonding, but instead are driven by the physical interactions described above and result in nanoparticles within the microsphere matrix. In addition, any combination of chemical or physical interactions described above can be used to incorporate radiopaque nanoparticles with microspheres.

In another example of method 12, shown as the middle path in FIG. 2, the RO material can be produced by creating a RO material and biodegradable linker complex. Linkers can include simple chemicals such as amines, carboxyl groups, or peptides, in addition to complex molecules such as proteins or functionalized polymers, and be biodegradable. After start at S20, the raw RO material 20 can be bound to a biodegradable linker 23 in step S22, creating a radiopaque material 22 configured as a radiopaque material-linker complex. The radiopaque material-linker complex can be produced in a suitable reactor with temperature and mixing control. As further discussed below with respect to FIG. 3, the RO material-linker complex 22 can then be incorporated with a microsphere particle, where the linker would biodegrade in vivo and release the radiopaque material from the microsphere to reduce the concentration for future treatments.

This example process can be used in cases where the microsphere material itself is not readily biodegradable. The radiopaque material-linker complex can be incorporated in or on the microsphere via conjugation chemistry between the linker functional groups and the microsphere functional groups to generate covalent bonding between the microsphere and linker. These functional groups can include hydrocarbons, halogenated groups, groups containing oxygen, groups containing nitrogen, groups containing sulfur, groups containing phosphorous, groups containing metals, or groups containing any other common chemical moiety on the microsphere and/or linker. Alternately, the radiopaque material-linker complex can be loaded into the microparticle (via encapsulation during microsphere formation) through physical interactions driven by charged group interactions, hydrophobic/hydrophilic interactions, hydrogen bonding, pi-pi stacking, or weaker forces such as Van der Waals. These interactions do not form covalent bonding, but instead are driven by the physical interactions described above and result in radiopaque linkers within the microsphere matrix. In addition, any combination of chemical or physical interactions described above can be used to incorporate radiopaque material-linker complexes with microspheres.

In another example of method 12, shown as the bottom path in FIG. 2, the RO material 22 can be produced as a metal-organic complex. In such examples, after start at S20, the RO material 22 can be created from the raw radiopaque material 20 and an organic chemical complex 24 in step S23. For example, a metal-organic complex can be created using metal-chloride and acetylacetone in chloroform, which is defined in a reactor with temperature and mixing control. As further discussed below with respect to FIG. 3, the metal-organic complex can then be incorporated into the microsphere via charge interactions between the microsphere material and the metal-organic molecule.

The radiopaque material-organic complex can be incorporated in or on the microsphere via conjugation chemistry between the metal-organic complex functional groups and the microsphere functional groups to generate covalent bonding between the microsphere and metal-organic complex. These functional groups can include hydrocarbons, halogenated groups, groups containing oxygen, groups containing nitrogen, groups containing sulfur, groups containing phosphorous, groups containing metals, or groups containing any other common chemical moiety on the microspheres and/or metal-organic complex. Alternately, metal-organic complex can be loaded into the microparticle (either via encapsulation during microsphere formation or through post-synthesis loading) through physical interactions driven by charged group interactions, hydrophobic/hydrophilic interactions, hydrogen bonding, pi-pi stacking, or weaker forces such as Van der Waals. These interactions do not form covalent bonding, but instead are driven by the physical interactions described above and result in metal-organic complex within the microsphere matrix. This method is seen in the example described above involving a metal-organic complex made using metal-chloride and acetylacetone in chloroform, which then interacts with the microsphere via opposite charges of the metal-organic complex and microsphere material. In addition, any combination of chemical or physical interactions described above can be used to incorporate radiopaque metal-organic complex with microspheres.

In FIG. 3, an example method 14 of combining the radiopaque material 22 with a biodegradable material are shown. The methods shown and described can be used, for example, at step S2 of method 10 (FIG. 1). As a result of method 14, unmodified radiopaque microspheres 33 are formed (i.e., microspheres containing RO material and a polymer without any post-synthesis modifications). The method 14 includes three pathways or optional sub-methods that can be used to produce the unmodified radiopaque microspheres 33. The RO microspheres 33 can be biodegradable and loaded with the RO material 22. A suitable biodegradable material such as natural polymers 31 can be selected. Examples include gelatin, alginate, chitosan, hyaluronic acid, collagen, cellulose, fibrinogen, fibrin, silk, elastin, hydroxyapatite, decellularized matrix, heparin, agarose, natural polysaccharides, and albumin. These materials can include modifications such as by conjugating moieties containing oxygen, nitrogen, carbons, halogens, sulfur, phosphorous, metals, or any other common chemical moiety, such as in the case of gelatin methacrylate, triacyl gelatin, and hyaluronic acid methacrylate.

Alternatively, a suitable biodegradable material such as synthetic polymers 34 derived from the general groups of polyesters, polyanhydrides, polyureas, synthetic polysaccharides, polyphosphazenes, and/or polyamides can be selected. Examples include poly(L-lactide) (PLLA), poly(lactic-co-glycolic acid) (PLGA), polyethylene glycol (PEG), polyurethane (PU), and polyvinyl alcohol (PVA). These materials can include modifications such as by conjugating moieties containing oxygen, nitrogen, carbons, halogens, sulfur, phosphorous, metals, or any other chemical moiety, such as in the case of PEG-diacrylate.

In an example, shown as the top path in FIG. 3, a method of producing a natural polymer microsphere production is provided. Unmodified RO-polymer microspheres 33 are defined using various processes. In this example step S31, an emulsion polymerization is used where the material is formed into microspheres and cross-linked by a cross-linker(s). A natural polymer material 31 in an aqueous solution is added to a solution containing oil, solvent, and surfactant 32. The solution is homogenized and allowed to mix. The cross-linker is added to the solution to cross link the natural polymer 31 and define unmodified RO-polymer microspheres 33. The reactor controls for size and properties by controlling, for example, temperature, mixing speed, time, washing, and solution concentrations. The RO materials can be introduced to the natural polymer through any suitable method including those described above. The emulsification reaction can be performed via water-in-oil (described above), oil-in-water, or any other type of emulsion method. For a specific example, synthesizing radiopaque alginate microspheres would use the same water-in-oil emulsion polymerization but with CaCl₂) as the cross-linker. Additional methods of microsphere synthesis can be employed including solvent evaporation or extraction, bulk polymerization, suspension polymerization, sedimentation polymerization, interfacial polymerization, phase separation co-acervation, ion-induced coagulation, and spray-drying.

In examples shown as the middle path and the bottom path in FIG. 3, a synthetic polymer microsphere synthesis is provided with biodegradable material, which can be defined using various processes. In a first example, shown as the middle path of FIG. 3, a microfluidic process may be used to combine the radiopaque material into a biodegradable microsphere. In such an example, a biodegradable material in an organic solvent 34 (also called the disperse phase) is added to a water solution containing surfactant (also called the continuous phase). The solution is homogenized and mixed to define unmodified RO-polymer microspheres 33, followed by solvent evaporation. In the microfluidic approach at step S32, the disperse phase can be pushed through microfluidic capillaries through the continuous phase to form uniform droplets of biodegradable material, which in turn form solid microspheres after solvent evaporation. The RO materials can be introduced to the natural polymer through any suitable method including those described above.

In another example, shown as the bottom pathway of FIG. 3 that includes step S33, the biodegradable radiopaque microspheres can be produced using polymer synthesis via emulsification. In such examples, a biodegradable material in an organic solvent 34 is added to a water solution containing surfactant. The solution is homogenized and mixed to define unmodified RO-polymer microspheres 33, followed by solvent evaporation. Like the natural polymer method (top pathway of FIG. 3) described above, the reactor used at step S33 can control size and physical properties of the unmodified RO-polymer microspheres 33 by controlling, for example, temperature, mixing speed, time, washing, and solution concentrations. The emulsification reaction can be performed via water-in-solvent (described above), solvent-in-water, or any other type of emulsion method. Additional methods of microsphere synthesis can be employed, including solvent evaporation or extraction, bulk polymerization, suspension polymerization, sedimentation polymerization, interfacial polymerization, phase separation co-acervation, ion-induced coagulation, and spray-drying.

As a result of method 14, unmodified or unfinished biodegradable radiopaque microspheres are formed. The microspheres, however, may not be ready for use during a treatment. The microspheres may have unsuitable size distributions, may include residual materials from the forming process that are unsuitable for treatment, or may otherwise need further processing.

In FIG. 4, an example method 16 of finishing the unmodified biodegradable radiopaque microspheres is provided (unmodified biodegradable radiopaque microspheres being those that have not undergone further chemical reactions to change surface properties, density, biodegradation, etc., if needed). The method 16 can be performed on the unmodified biodegradable radiopaque microspheres 33 of method 14, for example. The method 16 can describe example finishing methods or process that can be performed at step S3 of method 10, for example. In some examples, the unmodified biodegradable radiopaque microspheres 33 can undergo finish processing by washing, sieving, drying, and other processing at step S41. The unmodified RO-polymer microspheres 33 can undergo such processing to obtain microspheres of uniform size distribution and target properties.

Optionally, after S41, the microspheres can be modified in post-synthesis reactions S42 to adjust the surface properties, density, and biodegradation 41 to match the radioactive microsphere flow parameters and/or degradation needs. Surface modifications can include, but are not limited to, modifying chemical groups on the microsphere surface (through covalent conjugation chemistry methods to bind new groups to existing surface groups, such as by changing a nitrogen containing group to an oxygen containing group), extending polymer chains on the microsphere surface (such as by including a longer polymer chain around the microsphere), and/or coating the microsphere surface with different materials (such as coating the microsphere with SiO₂ if the radioembolization microsphere is made from SiO₂ as well). Density modifications can include incorporating heavier elements (such as metal-based radiopaque elements) into the microsphere increase density, or reacting the microsphere with additional chemicals to reduce density (such as by processing the microsphere in a basic solution to break down chemical bonds and allow for water infiltration). Biodegradation modifications can include incorporation of a linker as described in section 0035 (but without the RO element) or reacting the microsphere with additional chemicals to improve biodegradation (such as by processing the microsphere in a basic solution to break down chemical bonds and allow for water infiltration).

Step S42 may not be necessary depending on the radiopaque material and microsphere synthesis methods. For example, if tantalum nanoparticles are encapsulated within PLGA microspheres, the biodegradable radiopaque microsphere density may already match the radioactive microsphere density and, therefore, would not require any additional modification.

As can be appreciated, the methods 12, 14, and 16 can be combined to define a method to produce finished biodegradable radiopaque microspheres 44. The various pathways and/or sub-methods previously described can be used in various examples to provide biodegradable radiopaque microspheres 44 for use during treatment procedures.

Once suitable biodegradable radiopaque microspheres 44 are fabricated, they can be mixed with radioactive microspheres. The mixing can be performed in a ‘hot cell’ that should include a radioactive shield to protect operators and production personal from radiation exposure. The mixing can be performed by combining predetermined weight ratios of biodegradable radiopaque microspheres 44 and radioactive microspheres. The mixing can be followed by packaging and incorporation within a delivery device. The ratio of the biodegradable radiopaque microspheres 44 and the radioactive microspheres can be determined through experimental analysis, specifically in vivo analysis of the radioactive dosage required and the imaging signal requirements of the in-room imaging system. This determination is meant to provide a balance that provides for improved patient treatment and accurate imaging to correlate with radioactive dosimetry.

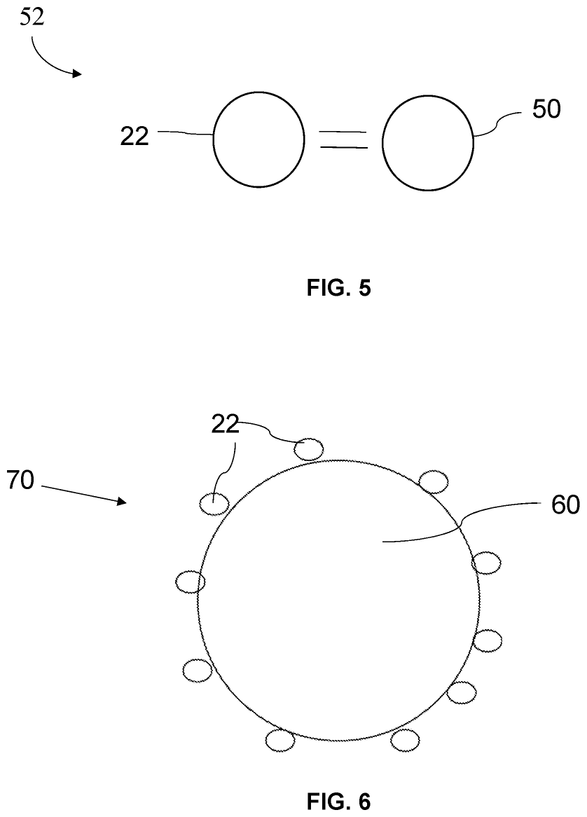

The biodegradable radiopaque microspheres of the present disclosure can include various configurations and/or structures that can be a result of the various production methods previously described. In some examples, the biodegradable radiopaque particles of the present disclosure can be formed by conjugating/cross-linking the radiopaque material to a biodegradable material. Such an example biodegradable radiopaque particle 52 is shown in FIG. 5. As shown the particle 52 can include a radiopaque material 22 linked to a biodegradable material 50, defined by conjugation/cross-linking. The particle 52, for example, can be formed using step S22 of method 12. A configuration using conjugation/cross-linking is also seen in a biodegradable radiopaque particle 70 in FIG. 6, where the biodegradable material 60 is covered with the radiopaque material 22. The biodegradable radiopaque particle 70 represents a configuration of the biodegradable radiopaque particle 70 generated by conjugation/cross-linking, where the radiopaque material 22 is much smaller than the biodegradable material 60. Alternatively, the biodegradable radiopaque particle 52 represents a case where the radiopaque material 22 and the biodegradable material 50 are similar in size.

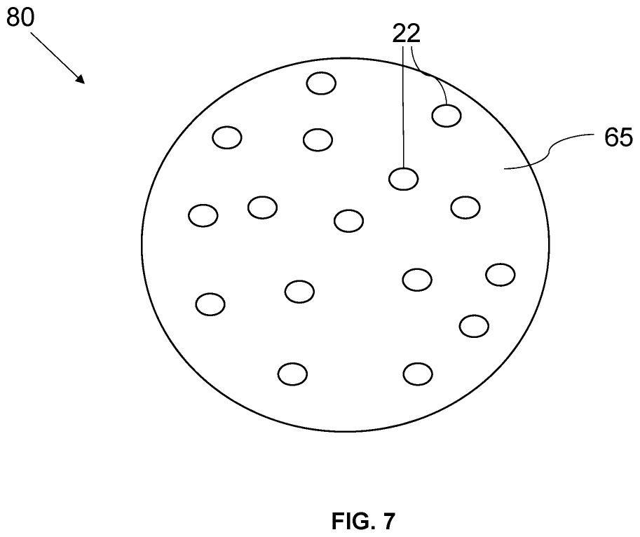

In another example, a biodegradable radiopaque particle can include a biodegradable material encapsulating a radiopaque material. One example biodegradable radiopaque particle 80 is shown in FIG. 7. In this example, the biodegradable radiopaque particle 80 can include a radiopaque material 22 encapsulated in a biodegradable material 65. Such a particle 80 can be produced via emulsification such as in steps S31 and S33 of method 14. In other examples, other biodegradable radiopaque particles can be formed in accordance with the present disclosure.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A microsphere comprising:
a radiopaque material, and
a biodegradable material.

2. The microsphere of claim 1, wherein the radiopaque material is a nanoparticle.

3. The microsphere of claim 1, wherein the biodegradable material is chemically bound to the radiopaque material.

4. The microsphere of claim 1, wherein the radiopaque material is encapsulated by the biodegradable material.

5. The microsphere of claim 1, wherein the biodegradable material is coated by the radiopaque material.

6. The microsphere of claim 1, wherein the biodegradable material includes at least one of gelatin, alginate, chitosan, hyaluronic acid, collagen, cellulose, fibrinogen, fibrin, silk, elastin, hydroxyapatite, decellularized matrix, heparin, agarose, natural polysaccharides, albumin, gelatin methacrylate, triacyl gelatin, and hyaluronic acid methacrylate.

7. The microsphere of claim 1, wherein the biodegradable material includes as least one of poly(L-lactide) (PLLA), poly(lactic-co-glycolic acid) (PLGA), polyethylene glycol (PEG), polyurethane (PU), and polyvinyl alcohol (PVA).

8. The microsphere of claim 1, wherein the radiopaque material includes at least one of tantalum, gold, barium, strontium, gallium or iodine which may be in the form of pure metals, metal oxides, alloys, compounds, or any other suitable chemical formulation.

9. A method of making a microsphere comprising linking a biodegradable material with a radiopaque material.

10. A method of making a microsphere comprising encapsulating a radiopaque material with a biodegradable material.

11. A method of making a microsphere comprising coating a biodegradable material with a radiopaque material.

12. A method of making microspheres comprising:
providing a radiopaque material;
combining the radiopaque material with a biodegradable material to define biodegradable radiopaque microspheres; and
finish processing the biodegradable radiopaque microspheres.

13. The method of claim 12, wherein the radiopaque material is provided by synthesizing radiopaque nanoparticles through addition of a surfactant to a raw radiopaque material.

14. The method of claim 12, wherein the radiopaque material is provided by creating a radiopaque material and a biodegradable linker complex.

15. The method of claim 14, wherein the linker complex includes at least one of an amine, a carboxyl group, a peptide, a protein or functional polymer, and is biodegradable.

16. The method of claim 12, wherein the radiopaque material is provided as a metal-organic complex.

17. The method of claim 12, wherein the finish processing includes obtaining biodegradable radiopaque microspheres of uniform size distribution and target properties.
